(19) United States
(12) Patent Application Publication
(10) Pub. No.: US 2005/0234424 A1
(43) Pub. Date: Oct. 20, 2005

(54) Pharmaceutical pig and method of use

(76) Inventors: Quent Besing, Eureka, MO (US); Joseph Angeloni, Peckville, PA (US); Michael Joseph Rossi, Mountaintop, PA (US); Andrew Williams, Lake St. Louis, MO (US)

Correspondence Address: Tim A Cheatham;Mallinckrodt Inc, 675 McDonnell Boulevard, PO Box 5840, St Louis, MO, 63134, US

(21) Appl. No.: 10/527,011

(22) PCT Filed: Sep. 24, 2003
(86) PCT No.: PCT/US03/30227

§ 371 Date: Mar. 7, 2005

(51) Int. Cl. A61M 31/00; G21C 1/01; A61N 5/00

(52) U.S. Cl. 604/500; 376/463; 600/1

(57) ABSTRACT

A pharmaceutical pig is used to transport a syringe containing a liquid radiopharmaceutical from a radiopharmacy to a medical facility for administration to a patient. The used syringe is sent back in the pig to the radiopharmacy for disposal. The pig includes a cap that is removably attached to a base. Shielding elements, typically formed from lead, are enclosed in the cap and the base. The pig can be used to transport a conventional syringe or a safety syringe. The pig can be used with or without an inner liner in the base. There is never an inner liner in the cap.

### BACKGROUND OF INVENTION

A pharmaceutical pig is used for transportation of liquid radiopharmaceuticals. A radiopharmacy typically dispenses a liquid radiopharmaceutical into a syringe, which is placed in a pharmaceutical pig for transport to a medical facility. The pig reduces unwanted exposure from the radioactive material and protects the syringe from damage. After delivery, the pig is opened, the syringe is removed and the radiopharmaceutical is administered to a patient. The used syringe is put back in the pig and returned to the radiopharmacy for disposal. Some radiopharmacies are independently owned and others are owned and operated in nationwide networks by Cardinal Health, Inc., having a place of business at 7000 Cardinal Place, Dublin, Ohio 43017 and Mallinckrodt Inc., a business of Tyco International, Ltd. The pharmaceutical pig of the present invention may be used with a conventional syringe or a safety syringe. The pharmaceutical pig of the present invention may be used with or without liners.

### DESCRIPTION OF RELATED ART

Conventional pharmaceutical pigs are used on a daily basis by radiopharmacies across the country. Many of the conventional pigs in current use are formed from plastic and lead. Of course, the lead is used as shielding material for the radiopharmaceutical. Conventional plastic/lead pigs are typically configured in a two-part or a three-part design, discussed in greater detail below. Other conventional pigs are formed from plastic and tungsten. The tungsten is an alternative shielding material to lead, but it is much more expensive.

U.S. Design Pat. No. 447,213 shows a plastic/tungsten pharmaceutical pig currently in production by Syncor International Corporation of Woodland Hills, Calif. (“Syncor”), which has now been acquired by Cardinal Health, Inc. Another tungsten pharmaceutical pig from Syncor is described in U.S. Pat. No. 5,828,073. Both of these patents describe elongate pigs contoured to transport a conventional syringe. Some radiopharmaceuticals, such as radioactive iodine, are typically dispensed into capsules or vials. These capsules or vials are sometimes transported in squat cylindrical containers such as the one described in U.S. Pat. No. 5,834,788. These squat cylindrical containers are sometimes also called pharmaceutical pigs.

However, most liquid radiopharmaceuticals are dispensed into a syringe. The present invention is a method and apparatus including a pharmaceutical pig used to transport syringes filled with a liquid radiopharmaceutical. Pharmaceutical pigs currently used with syringes are elongate devices sized to enclose a single syringe that holds a dose for a single patient. Conventional two-part pharmaceutical pigs are available from Biodex Medical Systems, Inc. of Shirley, N.Y. (“Biodex”) and are commonly used in the Mallinckrodt system of radiopharmacies. Conventional three-part pharmaceutical pigs are produced by Cardinal Health, Inc. and are shown in U.S. Pat. No. 5,519,931. These conventional three-part pharmaceutical pigs are believed to be in widespread use in the Cardinal Health, Inc. system of radiopharmacies to transport conventional syringes.

The Biodex two-part pig is formed from: a) an outer plastic shell having a removable plastic top that threadibly engages a plastic base; and b) an inner shield having an upper lead section that fits in the plastic top and a lower lead section that fits in the plastic base. Conventional syringes are transported in this two-part pig. However, because of the possibility of contamination, the lower section of the pig is washed and disinfected after each use in the Mallinckrodt system of radiopharmacies.

The Syncor three-part pharmaceutical pig disclosed in U.S. Pat. No. 5,519,931 is formed from the following components: a) an outer shell having a removable plastic top that threadibly engages a plastic base; b) an inner shield having an upper lead section that fits in the plastic top and a lower lead section that fits in the plastic base; and c) an inner disposable liner having a removable plastic cap that connects to a plastic base. A conventional syringe is contained in the disposable plastic liner, which fits into the lead portion of the pig. U.S. Pat. No. 5,672,883 (Re. 36,693) is an example of a disposable plastic liner. In Re 36,693, the liner is a self-contained biohazard container designed to capture any of the radiopharmaceutical that may inadvertently leak from a conventional syringe during transit. U.S. Pat. No. 5,672,883 (Re. 36,693) is a divisional of U.S. Pat. No. 5,519,931 and contains method of use claims for the three-part pig described above. U.S. Pat. No. 5,536,945 is another divisional of U.S. Pat. No. 5,519,931 and contains apparatus claims for the aforementioned inner liner, also referred to as a sharps container, having at least one resilient snap to keep the cap attached to the base. After the pig and the used syringe are returned to the radiopharmacy, the liner and the syringe are placed in a disposal container. Other pigs have also been developed.

John B. Phillips is listed as the inventor on several patents for a three-part pharmaceutical pig having: a) an outer plastic shell; b) an inner lead shield; and c) a removable inner liner to hold a syringe. The Phillips' patents are as follows: U.S. Pat. No. 5,611,429; U.S. Pat. No. 5,918,443; and U.S. Pat. No. 6,155,420. The removable inner liner in the Phillips' design has a flared hexagonal shaped section sized to surround the finger grip of the syringe and hold it securely in place during transit. These patents also disclose various ways, such as a detent, to securely hold the cap and the base of the inner liner together.

Conventional three-part lead/plastic pigs, such as the Syncor design or the Phillips design described above, rely on a removable inner liner having a cap and base to contain the syringe and prevent contamination of the lead shielding material with the radiopharmaceutical. However, both the two-part lead/plastic pig and the three-part lead/plastic pig have exposed lead on the interior. There is a need for a new design that protects the lead from inadvertent contamination by the liquid radiopharmaceutical.

Conventional three-part lead/plastic pigs have a radiation shield that is generally uniform in thickness. There is a need to reduce the weight of lead pigs and still retain the shielding capability of conventional designs. In U.S. Pat. No. 5,828,073, a pig, preferably made of tungsten employs a thin wall design near the needle and the plunger of the syringe. Another problem with some prior art designs is the point of abutment of the shielding material in the cap and the shielding material in the base, which may permit radiation leakage. Some prior art designs, such as U.S. Pat. No. 3,531,644 and U.S. Pat. No. 5,611,429 provide for overlap in the shielding material to reduce radiation leakage.

Many conventional three-part lead/plastic pigs use a threaded design to connect the cap and the base. Some of these prior art designs require several turns to connect the cap and the base. In a busy radiopharmacy, there is a need for a faster and easier way to attach the cap to the base.

A label is attached to the pharmaceutical pig at the radiopharmacy prior to the transport to the hospital. The label contains important information including: the patient's name; the type of radiopharmaceutical; the dose; the name of the hospital and the address; among other things. These labels are typically attached to conventional pigs with adhesives or rubber bands. Some radiopharmacies may use several hundred pigs per day. If the labels are secured by adhesives, they must be removed, which is time consuming and tedious. If rubber bands are used, they may break or obscure important information from the view of medical personnel. There is a need for a better way to attach and remove the label from the pig. One prior art attempt to deal with labeling issues is U.S. Pat. No. 5,545,139.

The revised OSHA Bloodborne Pathogens Standard (29 CFR 1910.1030) went into effect in 2001. The Standard requires healthcare facilities under the jurisdiction of OSHA to use safer medical devices, such as sharps with engineered sharps injury protection and needleless systems. This Standard and other OSHA directives require an annual review of a facility's exposure control plan and the use of safer medical devices to help reduce needle sticks and other sharps injuries. The Needlestick Safety and Prevention Act, these federal rules and regulations as well as other state rules and regulations have encouraged the development of “safety syringes” such as the Monoject® Safety Syringe from Kendall Company LP of Mansfield, Mass., The Safety-Lok from Becton-Dickinson and Company of Franklin Lakes, N.J. (“B-D”) and the SafeSnap® from U.S. Medical Instruments of San Diego, Calif.

Operationally, these safety syringes function in different ways, but the purpose is to keep the tip of the needle out of contact with healthcare personnel before and after a medication has been administered to a patient. For example, the Monoject® Safety Syringe has an extendable outer tubular sheath that moves from a retracted position around the barrel to an extended locked position that surrounds the needle after it has been used. Safety syringes from B-D also have an extendable outer tubular sheath that moves from a retracted position to an extended locked position surrounding the needle. Other designs have retractable needles, such as the NMT available from New Medical Technology, Inc of Zionsville, Ind. (U.S. Pat. No. 5,782,804). The SafeSnap® safety syringe also has a retractable needle that can be moved from an extended position to a retracted position in the barrel after it has been used. A portion of the plunger can then be snapped off and inserted in the open end of the barrel to create a self-contained biohazard container. One or more of the following U.S. patents may cover the SafeSnap® syringe: U.S. Pat. No. 4,710,170; U.S. Pat. No. 5,205,824; U.S. Pat. No. 5,308,329 and U.S. Pat. No. 5,401,246. Other devices use different designs to shield the needle after it has been used. For example, the SafetyGlide® needle from B-D has a stainless steel latch that shields the needle after activation. (U.S. Pat. No. 5,348,544). The SafetyGlide® needle can be used with any conventional syringe to convert it to a safety syringe.

The term “safety syringe” as used herein includes any of the following products and any other syringes with a needle, not listed below that have an apparatus to protect healthcare personnel from accidental needle stick:

Extendable Sheath Designs.

Monoject® Safety Syringe from Kendall Company LP of Mansfield, Mass.

Safety Lok® syringe from B-D of Franklin Lakes, N.J.

Gettig Guard® syringe from Gettig Pharmaceutical Instrument Co. of Spring Mill, Pa.

Univec Sliding Sheath® syringe from Univec of Farmingdale, N.Y.

Retractable Needle Syringes.

SafeSnap® syringe from U.S. Medical Instruments of San Diego, Calif.

NMT™ syringe from New Medical Technology, Inc. of Zionsville, Ind.

Elite safety syringe from Medi-Hut Co., Inc of Lakewood, N.J.

Vanish Point® syringe from Retractable Technologies of Lewisville, Tex.

Needle Guards That Can Be Used With Any Syringe.

SafetyGlide® needle from Becton-Dickson and Company of Franklin Lakes, N.J.

Sterimatic™ safety needle from Sterimatic Medical Corp. of the United Kingdom.

Needle-Pro™ from SIMS Portex, Inc. of Keene, N.H. (Hinged recap) Miscellaneous Designs.

Protector Syringe and Safety Cap System from InjectiMed, Inc. of Ventura, Calif.

U.S. Pat. No. 6,425,174, assigned to Syncor International Corp. discloses the use of a separate sharps container 12 for holding a standard syringe 14 within a radiopharmaceutical pig 10. The sharps container 12 is in the form of a tubular housing.

To the applicant's knowledge, “safety syringes” have never been used to transport liquid radiopharmaceuticals in pharmaceutical pigs from a radiopharmacy to a medical facility. In one embodiment, the present invention combines an improved pharmaceutical pig with a “safety syringe” to transport liquid radiopharmaceuticals from a radiopharmacy to a medical facility.

### SUMMARY OF INVENTION

A pharmaceutical pig is sized and arranged to transport a single syringe containing a unit dose of a radiopharmaceutical from a radiopharmacy to a medical facility such as a doctor's office, clinic or hospital. After the radiopharmaceutical has been administered to a patient, the used syringe is put back into the pig and returned to the radiopharmacy for proper disposal. The present invention can be configured in a two-part or a three-part design. The present invention may be used with conventional syringes or safety syringes.

In the two-part design, the present invention includes: a) an elongate stainless steel cap removably attached to a base; and b) inner shielding elements. The elongate cap has an inner and outer stainless steel shell to completely enclose a cap shielding element, which is typically formed from lead. The elongate base has an inner and outer stainless steel shell to completely enclose the base shielding element, which is also typically formed from lead. The shielding elements are completely enclosed and protected from inadvertent contamination from a liquid radiopharmaceutical by the stainless steel shells. A bayonet closure removably attaches the cap to the base.

In the two-part design of the present invention, a conventional syringe or a safety syringe may be used to contain the radiopharmaceutical. In one embodiment, a sleeve, at least a portion of which is transparent, slips on and off at least a portion of the base to removably secure a label to the base. No adhesive or rubber bands are needed as in conventional designs. The base shielding material is tapered near the needle end to reduce the overall weight of the pig. Furthermore, the cap shielding element overlaps the base shielding element when the cap is connected to the base. This overlap reduces radiation leakage from the pig at the point the cap and base are joined together.

In the three-part design, the present invention includes: a) an elongate stainless steel cap removably attached to a base; b) inner shielding elements; and c) a lower inner liner in the base. A conventional syringe or a safety syringe may be used with this three-part design. Unlike the prior art, there is never an inner liner in the cap of the present invention. The lower inner liner may have a test tube-like shape with a slight flair at the open end or it may have a straight wall. The inner liner may have a slight bead near the open end to achieve an interference fit with the base.

### BRIEF DESCRIPTION OF DRAWINGS

FIG. 1 is a section view of the cap shielding element separated from the base shielding element.

FIG. 2 is a section view of the elongate cap having an inner and outer shell separated from the elongate base having an inner and outer shell. There is no syringe or transparent flexible sleeve shown in this view.

FIG. 3 is a perspective view of the elongate cap separated from the elongate base. There is no transparent flexible sleeve shown in this view.

FIG. 4 is a section view of the assembled two-part pharmaceutical pig with the transparent flexible sleeve. There is no syringe shown in this view.

FIG. 5 is a perspective view of the transparent flexible sleeve.

FIG. 6 is a perspective view of the assembled two-part pharmaceutical pig of FIG. 4 with a label positioned under the transparent flexible sleeve.

FIG. 7 is an enlargement of the label from FIG. 6.

FIG. 8 is a plan view of the elongate cap.

FIG. 9 is a plan view of the elongate cap with screws inserted in the keyhole-shaped slots of the cap.

FIG. 10 is a plan view of the elongate cap of FIG. 9, except the cap and the base have been rotated counter-clockwise to lock the screws in the recesses of the keyhole-shaped slots.

FIG. 11 is a plan view of the screw retaining ring.

FIG. 12 is a section view of the assembled two-part pharmaceutical pig of FIG. 4 and a Monoject® Safety Syringe positioned inside the pig. This embodiment does not have an inner liner.

FIG. 13 is a section view of the assembled two-part pharmaceutical pig of FIG. 4 and a B-D safety syringe positioned inside the pig. This embodiment does not have an inner liner.

FIG. 14 is a section view of the assembled two-part pharmaceutical pig of FIG. 4 and a conventional syringe positioned inside the pig. This embodiment does not have an inner liner.

FIG. 15 is a section view of the base used in the three-part pharmaceutical pig. This embodiment includes a test tube-shaped inner liner with a flange at the open end.

FIG. 16 is a section view of the test tube-shaped inner liner of FIG. 15 with a flange at the open end.

FIG. 17 is a section view of the base used in the three-part pharmaceutical pig. This alternative embodiment includes an inner liner with straight sides at the open end.

FIG. 18 is a section view of the inner liner of FIG. 17 with straight sides at the open end.

FIG. 19 is a section view of the base used in the three-part pharmaceutical pig. This second alternative embodiment includes an inner liner with an interference fit bead near the open end.

FIG. 20 is a section view of the inner liner of FIG. 19 with an interference fit bead near the open end.

FIG. 21 is a section view of the three-part pharmaceutical pig with a Monoject® Safety Syringe and inner liner of FIGS. 15 and 16. In alternative configurations, not shown, the three-part pharmaceutical pig could be configured with the inner liner with straight sides of FIGS. 17 and 18 or the inner liner with a bead of FIGS. 19 and 20.

FIG. 22 is a section view of the three-part pharmaceutical pig with a conventional syringe and inner liner of FIGS. 15 and 16. An alternative configuration, not shown, could include the three-part pharmaceutical pig configured with the inner liner with straight sides of FIGS. 17 and 18 or the inner liner with a bead of FIGS. 19 and 20.

### DETAILED DESCRIPTION

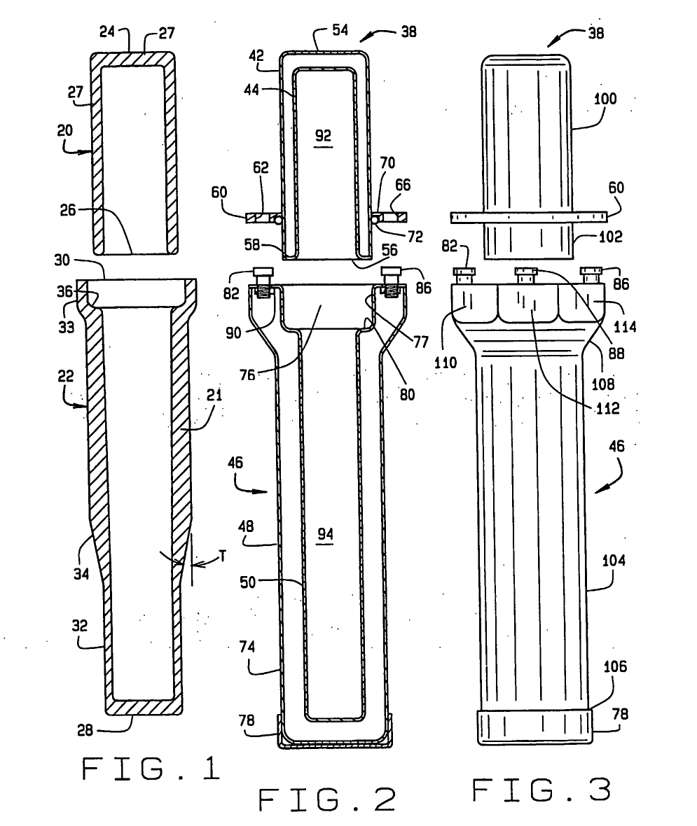

FIG. 1 is a section view of the cap shielding element 20 and the base shielding element 22. These shielding elements are typically formed from lead because it is relatively inexpensive and easy to form. These shielding elements can be formed from any material that blocks the radiation from the radiopharmaceutical. For example, tungsten is a suitable shielding element, but it is more expensive than lead and more difficult to form. Metallic-filled polymer composite materials such as the ECOMASS® compounds produced by Engineered Materials, an M.A. Hanna Company in Norcross, Ga. can also be used as shielding material.

The cap shielding element 20 has a closed end 24 and an open end 26. The walls 27 of the cap shielding element are of generally uniform thickness. The base shielding element 22 has a closed end 28 and an open end 30. The wall 32 of the base shielding element 22 near the closed end 28 is thinner than the wall 21. The thin wall 32 is to reduce the overall weight of the pharmaceutical pig. When assembled, the thin wall 32 is near the needle end 23 of a syringe, better seen in FIG. 12 and the thicker wall 21 is near the barrel end 25 of a syringe. The walls 27 of the cap shielding element 20 are thinner than the walls 21 in the base 22. In other words, the walls 21 that generally surround the barrel end 25 of the syringe are thicker than the walls 32 that generally surround the needle end 23 of the syringe and the walls 27 that are proximate a portion of the plunger of the syringe. The base shielding element has a taper 34 which forms an acute angle T when measured against the inside wall of the base shielding element 22. The base shielding element 22 has an enlarged section 33 near the open end 30 that forms a shoulder 36.

FIG. 2 is a section view of the elongate cap, generally identified by the numeral 38 having an outer shell 42 and an inner shell 44 and an elongate base 46 having an outer shell 48 and an inner shell 50. The outer shell 42 of the cap, the inner shell 44 of the cap, the outer shell 48 of the base and the inner shell 50 of the base are formed from stainless steel, although other suitable metals or plastics could also be used. In FIG. 2, the elongate cap 38 is detached from the elongate base 46.

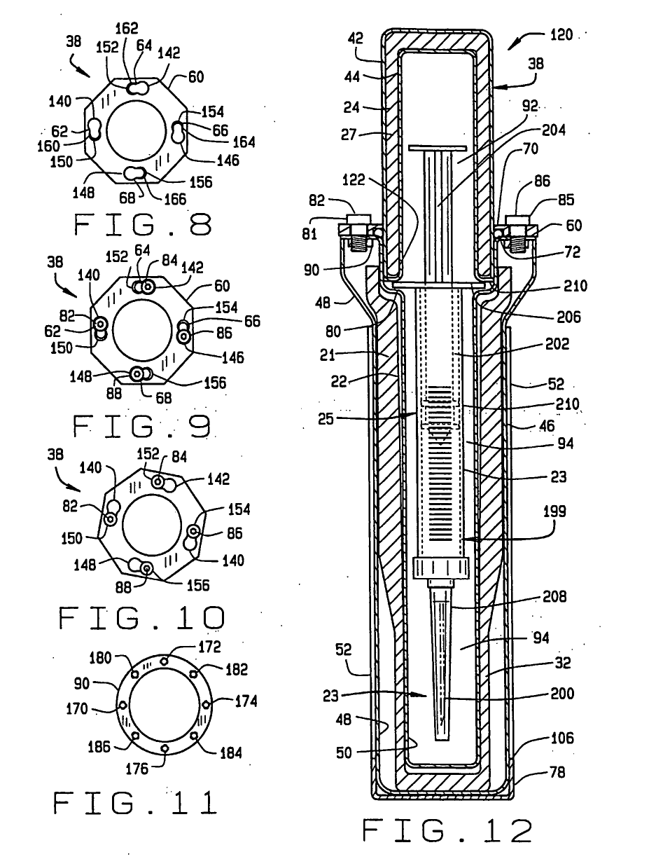

The elongate cap 38 has a closed end 54 and an open end 56. The outer shell 42 is welded to the inner shell 44 at 58 to hermetically enclose the cap shielding element 20. A flange 60 is formed on the cap 38 and has a plurality of keyhole-shaped apertures 62, 64, 66 and 68 formed therein. These apertures are better seen in FIG. 8. The outer shell 42 and the flange 60 define an o-ring channel 70. The o-ring channel 70 is sized and shaped to receive an o-ring 72.

The elongate base 46 has a closed end 74 and an open end 76. An end cap 78 is attached to the closed end 74. The inner shell 50 is enlarged near the open end 76 to define a shelf 80. A plurality of screws, 82, 84, 86 and 88, better seen in FIG. 9, pass through holes in the inner shell 50 and threadably engage a retainer ring 90. FIG. 11 is a plan view of the retainer ring 90.

The cap 38 has a hollow center section 92 sized to surround at least a portion of the plunger of the syringe, better seen in FIG. 12. The base 46 has a hollow center section 94 sized to generally surround the needle end 23 and at least a portion of the barrel end 25 of the syringe, better seen in FIG. 12.

FIG. 3 is a perspective view of the elongate cap 38 separated from the elongate base 46. The flange 60 divides the cap 38 into an upper section 100 and a lower section 102.

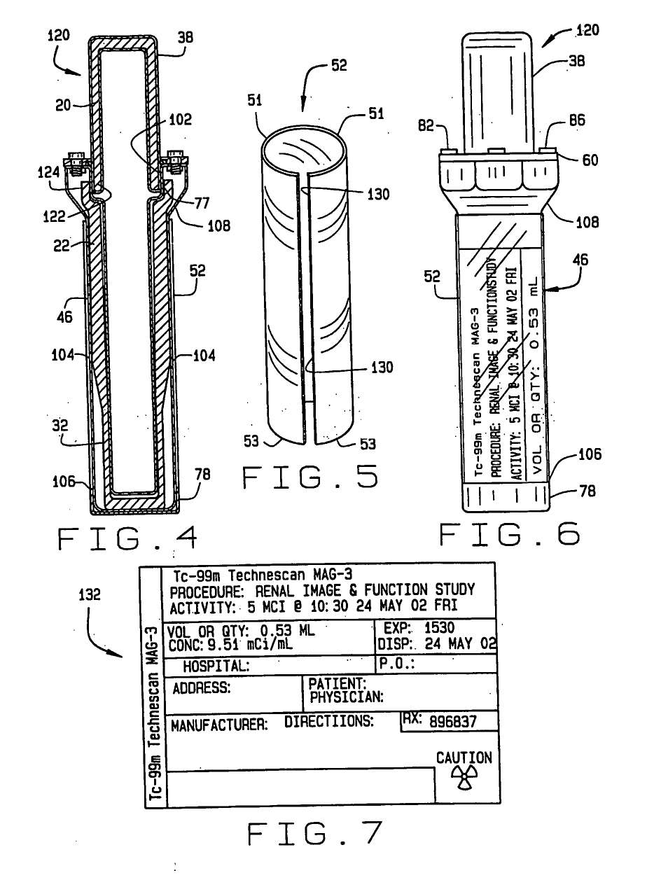

The base 46 includes a generally cylindrical section 104. The end cap 78 defines a shoulder 106 on the base 46. The transparent flexible sleeve 52, better seen in FIGS. 4-6 slips over the end cap 78 and grips the cylindrical section 104. The label 132 in FIG. 7 is captured between the flexible sleeve 52 and the cylindrical section 104 of the base 46.

The base 46 flares outward and forms an enlarged neck 108 that defines a plurality of anti-roll flats 110, 112 and 114. The screws 82, 84, 86 and 88 protrude above the enlarged neck 108 of the base 46. In FIG. 3 the cap 38 is disconnected from the base 46. In FIGS. 4 and 6, the cap 38 is connected to the base 46. The closure structure that connects the cap to the base is a bayonet design that includes the o-ring 72, the screws 82, 84, 86 and 88 and the keyhole-shaped apertures 62, 64, 66 and 68 in the flange 60. Other closure structures such as threads can also be used instead of the bayonet design to connect the cap 38 to the base 46. The transparent flexible sleeve 52 is not shown in FIGS. 1-3.

FIG. 4 is a section view of the assembled two-part pharmaceutical pig 120 with the transparent flexible sleeve 52 positioned around the cylindrical section 104 of the base 46. One end 53 of the sleeve 52 abuts the shoulder 106 formed by the end cap 78. The other end 51 of the sleeve is captured by the enlarged neck 108. The shoulder 106 and the enlarged neck 108 keep the flexible sleeve 52 from slipping off the pig 120. There is no syringe shown in this view. The cap 38 has been attached to the base 46 by the closure structure discussed above.

The lower section 102 of the cap 38 nests in an enlarged area 77 of the base 46. A portion 122 of the cap shielding element 20 overlaps a portion 124 of the base shielding element 22 to reduce radiation leakage from the pig 120.

FIG. 5 is a perspective view of the flexible sleeve 52. The sleeve 52 has a slit 130 that runs the entire length of the cylindrical sleeve. The sleeve can be formed from transparent plastic or other clear flexible materials that allow the sleeve to slip over the base 46. The purpose of the sleeve is to attach the label 132 of FIG. 7 to the pig 120 without the need for adhesives. At least a portion of the sleeve should be transparent to facilitate reading of the label.

FIG. 6 is a perspective view of the assembled two-part pharmaceutical pig 120 of FIG. 4 with a label 132 positioned under the transparent flexible sleeve 52. The clear sleeve 52 allows healthcare personnel to read the label 132.

FIG. 7 is an enlargement of the label 132 of FIG. 6. The format of the label 132 and the exact contents of the label will vary from one radiopharmacy to the next. The label 132 contains a description of the radiopharmaceutical, Tc-99m Technescan MAG-3, on the left-hand margin and the top. The label also contains the procedure that will be conducted with the radiopharmaceutical, i.e. renal image and function study. The activity of the radiopharmaceutical is also listed: 5 mCi at 10:30 24 May 2002.

The label 132 also contains the volume of the radiopharmaceutical, 0.53 milliliters, and the concentration, 9.51 mCi/mL. The expiration time, 1530 (3:30 PM) and the dispensing date, May 24, 2002 are also included.

The name and address of the hospital or medical facility to which the pig will be delivered is also listed on the label, but because of space requirements, this information has been omitted from FIG. 7. The name of the patient and physician are commonly listed on the label but have also been omitted from this figure. The label typically includes the name and address of the radiopharmacy that has filled the prescription and the prescription number, i.e. 896837.

The label contains a radioactive material warning symbol and may contain a statement that the U.S. Nuclear Regulatory Commission has approved distribution to this radiopharmaceutical to persons licensed to use by-product material listed in Paragraph 35.2000 of CFR Part 35 and to persons holding a equivalent license issued by an appropriately authorized authority. Some of this information has been omitted from FIG. 7 because of space constraints. Additional information may also be placed on the label such as the manufacturer, the invoice number and other data.

In some of the Mallinckrodt radiopharmacies, the first thing that is done after a prescription has been telephoned is to transcribe the information and enter it into a computer system, which is then followed by the printing of several labels. The first label is similar to the one shown in FIG. 7, which is currently attached to a pharmaceutical pig with a self-adhesive on the back of the label. A second label is printed concurrently and is attached to an empty conventional syringe. The syringe label, not shown contains some, but not all, of the information on the label 132 in FIG. 7. A third label is printed which is identical to the label 132, except it also has a bar code on the right hand side and is for internal use by the hospital or medical facility.

FIG. 8 is a plan view of the elongate cap 38. The flange 60 has a plurality of keyhole-shaped apertures 62, 64, 66 and 68. The large end 140, 142, 146 and 148 of each aperture is sized to allow the head of each screw to pass through. The small end of each aperture, 150, 152, 154 and 156 will not allow the head of the screws to pass through. A slight recess 160, 162, 164 and 166, better seen in FIG. 8, is formed at the small end of each aperture and is sized to receive and lock the head of each screw when the cap and the base are connected.

The closure structure operates as follows. First, the removable cap 38 is placed on the base 46 and the keyhole-shaped apertures 62, 64, 66 and 68 are aligned with the screws, 82, 84, 86 and 88. The heads of the screws then slip through the large ends 140, 142, 146 and 148 of each keyhole-shaped aperture as shown in FIG. 9. The cap 38 and the base 46 are then rotated counter-clockwise so the heads of the screws move to the small ends 150, 152, 154 and 156 of each keyhole-shaped aperture as shown in FIG. 10. The o-ring 72 is compressed when the cap 38 is attached to the base 46 and the o-ring 72 acts like a spring urging the cap away from the base. The spring action of the o-ring 72 causes the heads of the screws to lock into the recesses 160, 162, 164 and 166 formed on the small ends 150, 152, 154 and 156 of the keyhole-shaped apertures. Other closure structures are within the scope of this invention, such as conventional threads.

FIG. 11 is a plan view of the screw retaining ring 90. The ring 90 has a plurality of threaded holes, 170, 172, 174 and 176 that respectively engage the screws 82, 84, 86 and 88. Additional threaded holes, 180, 182, 184 and 186 are also formed in the ring 90. The additional holes are also sized to threadably engage the screws 82, 84, 86 and 88. The additional holes are formed in the ring 90 in case one or more of the original holes 170, 172, 174 or 176 becomes stripped or otherwise fails to function. In this situation, the screws are removed from the ring 90 and it is rotated with a probe or other long thin instrument about 45 degrees so that the additional holes 180, 182, 184 and 186 are realigned to receive the screws. In other words, the additional holes are a redundant feature that, under some circumstances, may allow for repair of the base without having to cut it apart.

FIG. 12 is a section view of the assembled two-part pharmaceutical pig 120 of FIG. 4 and a Monoject® Safety Syringe 199 is positioned inside the pig. The Mallinckrodt system of radiopharmacies uses syringes that hold different volumes of fluid, i.e. 1 cc, 3 cc, 5 cc, 6 cc, 10 cc and 12 cc. The pig therefore can be sized to accommodate syringes of different size and volume or in the alternative, pigs of different size can be used to accommodate syringes of different size and volume. For example, one size pig could accommodate the smaller syringes of 1-6 cc volume. Another size pig could accommodate the 10 and 12 cc syringes. In FIG. 12, the safety syringe 199 has a needle 200, shown in phantom, a barrel 202, also shown in phantom, a plunger 204, and finger grips 206, sometimes called wings. The finger grips 206 may be hexagonal, circular or polygonal; they may fully or partially surround the barrel 202. A removable needle cover 208 protects the needle 200, shown in phantom.

The finger grips 206, are captured between the shelf 80 formed in the inner shell 50 and the terminus 210 of the cap 38. The syringe is therefore prevented from lateral movement inside the pig 120 during transit. The needle 200, shown in phantom, and at least a portion of the barrel 202, shown in phantom, are positioned in the hollow center section 94 of the base 46. At least a portion of the plunger 204 is positioned in the hollow center section 92 of the cap 38.

The screw 82 threadably engages the hole 170 in the retaining ring 90, as shown in FIGS. 9, 10, 11 and 12. The head 81 of screw 82 is locked in the recess 160 of the small end 150 of the keyhole-shaped aperture 62. The screw 86 threadably engages the hole 174 in the retaining ring 90. The head 85 of screw 86 is locked in the recess 164 of the small end 154 of the keyhole-shaped aperture 66. The o-ring 72 is under compression and acts as a spring urging the cap 38 away from the base 46.

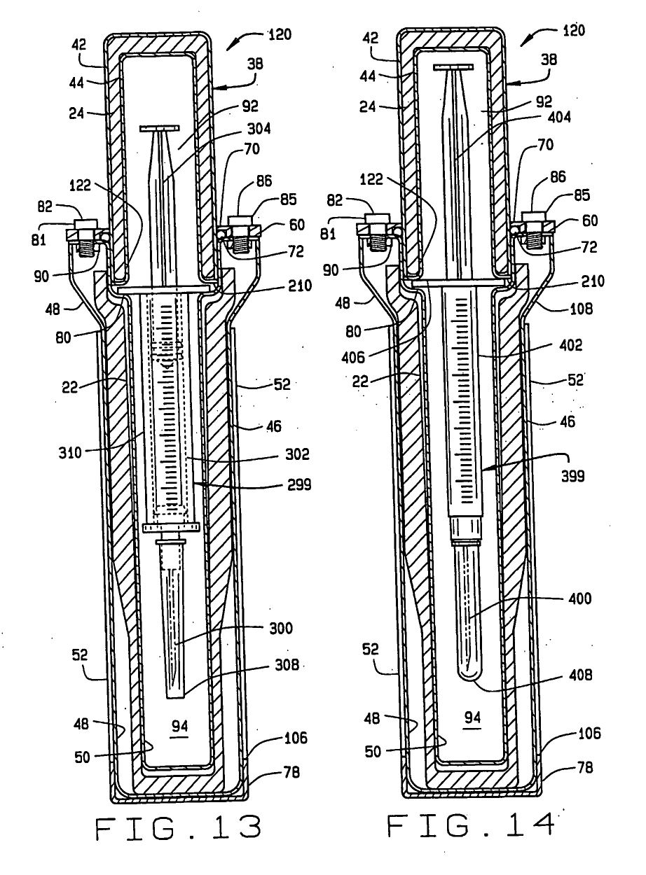

FIG. 13 is a section view of the assembled two-part pharmaceutical pig 120 of FIG. 4 and a B-D safety syringe 299 positioned inside the pig. This embodiment does not have an inner liner. The B-D safety syringe 299 has a needle 300, shown in phantom, a barrel, 302 also shown in phantom, a plunger 304 and finger grips 306, sometimes called wings. The finger grips 306 may be hexagonal, circular or polygonal; they may fully or partially surround the barrel 302. A removable needle cover 308 protects the needle 300, shown in phantom.

The finger grips 306 are captured between the shelf 80 formed in the inner shell 50 and the terminus 210 of the cap 38. The syringe 299 is therefore prevented from lateral movement inside the pig 120 during transit. The needle 300 and at least a portion of the barrel 302 are positioned in the hollow center section 94 of the base 46. At least a portion of the plunger 304 is positioned in the hollow center section 92 of the cap 38. An extendable outer tubular sheath 310 surrounds the barrel 302 in this view. The sheath 310 can be moved from the retracted position, shown in this figure to an extended position surrounding the needle after it has been used.

FIG. 14 is a section view of the assembled two-part pharmaceutical pig 120 of FIG. 4 and a conventional syringe 399 positioned inside the pig. This embodiment does not have an inner liner. The conventional syringe 399 has a needle 400, shown in phantom, a barrel 402, and a plunger 404 and finger grips 406, sometimes called wings. The finger grips 406 may be hexagonal, circular or polygonal; they may fully or partially surround the barrel 402. A removable needle cover 408 protects the needle 400, shown in phantom.

The finger grips 406 are captured between the shelf 80 formed in the inner shell 50 and the terminus 210 of the cap 38. The conventional syringe 399 is therefore prevented from lateral movement inside the pig 120 during transit. The needle 400 and at least a portion of the barrel 402 are positioned in the hollow center section 94 of the base 46. At least a portion of the plunger 404 is positioned in the hollow center section 92 of the cap 38.

FIG. 15 is a section view of the base 46 used in the three-part pharmaceutical pig 121 of FIG. 19. This first embodiment of the three-part pig includes a removable test tube-shaped inner liner 220 with a flared lip 232. The inner liner 220 has a closed end 226 and an open end 224. Unlike the prior art, there is never an inner liner in the cap 38.

The inner liner 220 with a flared lip 232 fits in the hollow center section 94 of the base 46. The flared lip 232 of the inner liner 220 is flush with the shelf 80, as shown in FIG. 15. The flared lip 232 may alternatively protrude slightly above the shelf 80.

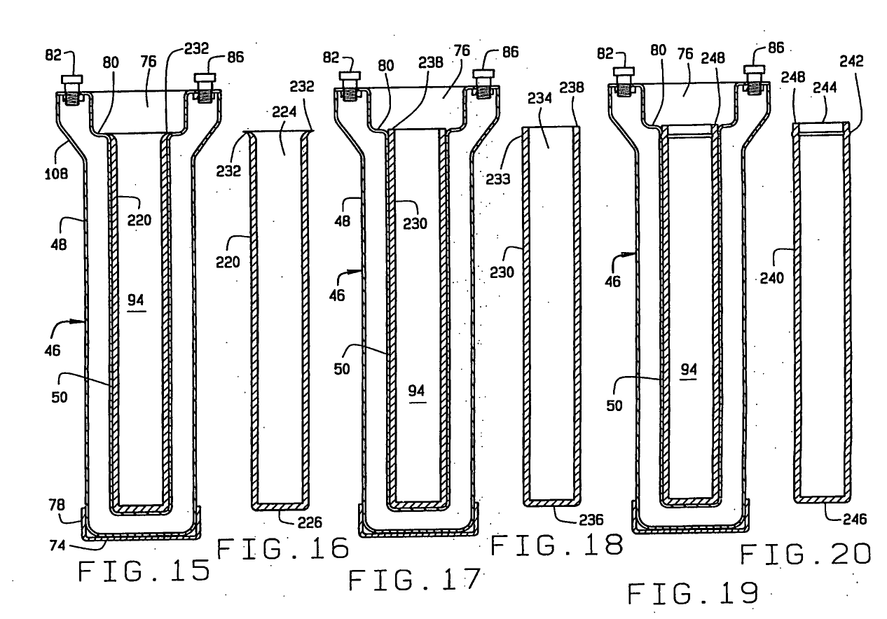

FIG. 16 is a section view of the removable test tube-shaped inner liner 220 of FIG. 15 with a flared lip 232 at the open end 224. The inner liner 220 can be formed from glass, plastic or any other liquid impermeable material. The inner liner 220 is intended to be disposable, but it could be removed, washed and reused if desired.

FIG. 17 is a section view of the base 46 used in the three-part pharmaceutical pig. The inner liner 230 has a closed end 236 and the open end 234. This second embodiment of the three-part pig includes an inner liner 230 with straight sides 233 at the open end 234. The inner liner 230 fits in the hollow center section 94 of the base 46. The top 238 of the inner liner 230 is flush with the shelf 80, as shown in FIG. 17. The top 238 may alternatively protrude slightly above the shelf 80. Unlike the prior art, there is never an inner liner in the cap 38.

FIG. 18 is a section view of the inner liner 230 of FIG. 17 with straight sides 233 at the open end 234. The inner liner 230 can be formed from glass, plastic or any other liquid impermeable material. The inner liner 230 is intended to be disposable, but it could be removed, washed and reused if desired.

FIG. 19 is a section view of the base 46 used in the three-part pharmaceutical pig. This third embodiment of the three-part pig includes an inner liner 240 with an interference fit bead 242. The inner liner 240 has a closed end 246 and on open end 244. In this third embodiment, the inner liner 240 is still removable, but the bead 242 is intended to more securely position the liner 240 in the hollow center section 94. The inner liner 240 fits in the hollow center section 94 of the base 46. The top 248 of the inner liner 240 is flush with the shelf 80, as shown in FIG. 19. The top 248 may alternatively protrude slightly above the shelf 80. Unlike the prior art, there is never a liner in the cap 38.

FIG. 20 is a section view of the inner liner 240 of FIG. 19 with an interference fit bead 242 near the open end 244. The inner liner 240 can be formed from glass, plastic or any other liquid impermeable material. The inner liner 240 is intended to be disposable, but it could be removed, washed and reused if desired.

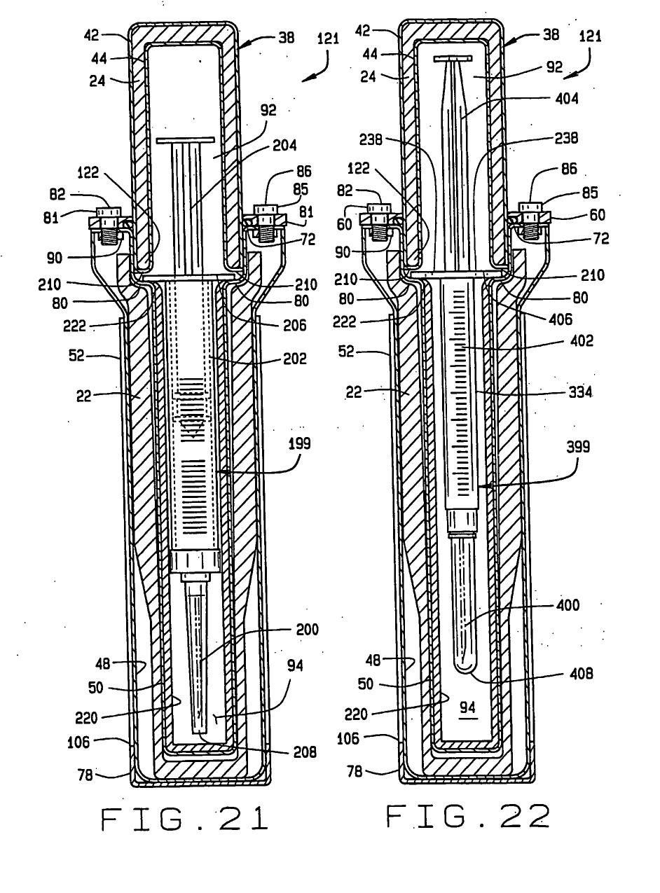

FIG. 21 is a section view of the three-part pharmaceutical pig with a Monoject® Safety Syringe 199 and inner liner 220 of FIGS. 15 and 16. The safety syringe 199 has a needle 200, shown in phantom, a barrel 202, also shown in phantom, a plunger 204, and finger grips 206, sometimes called wings. The finger grips 206 may be hexagonal, circular or polygonal; they may fully or partially surround the barrel 202. A removable needle cover 208 protects the needle 200, shown in phantom.

The finger grips 206, are captured between the shelf 80 formed in the inner shell 50 and the terminus 210 of the cap 38. The syringe is therefore prevented from lateral movement inside the pig 120 during transit. The needle 200, shown in phantom, and at least a portion of the barrel 202, shown in phantom, are positioned in the hollow center section 94 of the base 46. At least a portion of the plunger 204 is positioned in the hollow center section 92 of the cap 38.

In an alternative configuration, not shown, the three-part pharmaceutical pig could be configured with the inner liner with straight sides 230 of FIGS. 17 and 18 or the inner liner 240 with a bead of FIGS. 19 and 20. After a safety syringe has been used, it may be contaminated with the patient's blood and there may be a residual amount of radioactive liquid remaining in the needle or the barrel. For these reasons, an inner liner 220 is added to the two-part pig 120 thus converting it into a three-part pig 121. The inner liner 220 is designed to contain any bodily fluids and residual radiopharmaceuticals that may inadvertently leak from a used safety syringe.

For convenience, the inner liner is intended to be disposed after each use. Regulations require that pharmaceutical pigs be inspected and cleaned as necessary after each use. The Mallinckrodt system of radiopharmacies currently wash the lead/plastic Biodex pharmaceutical pigs after each use. It is contemplated that the system of Mallinckrodt will continue to wash the present invention after each use. Apparently, the Cardinal Healthcare Ltd. system of radiopharmacies does not wash their lead/plastic pigs after each use.

FIG. 22 is a section view of the three-part pharmaceutical pig 121 with a conventional syringe 399 and inner liner 220 of FIGS. 15 and 16. The three-part pharmaceutical pig 121 of FIG. 22 is the same as the two-part pig 120 shown in FIG. 12, with two differences. The first difference is the addition of an inner liner 220. The inner liner 220 is positioned in the base 46, never the cap 38. The second difference is the syringe. A conventional syringe 399 is used in the two-part pig 120 of FIG. 14. A conventional syringe 399 is shown in the three-part pig 121 of FIG. 22.

The inner liner can have many different shapes, only three of which are shown in the drawings. The first embodiment of the liner 220 has a test tube-like shape with a flared lip 232 and is shown in FIGS. 15, 16 and 21. The second embodiment of the liner 230 has straight sides and is shown in FIGS. 17 and 18. The third embodiment of the liner 240 has a bead near the open end and is shown in FIGS. 19 and 20. All three of these liners, 220, 230 and 240 can be used in the three-part pig 121.

As also shown in FIG. 22, the conventional syringe 399 has a needle 400, shown in phantom, a barrel 402, and a plunger 404 and finger grips 406, sometimes called wings. The finger grips 406 may be hexagonal, circular or polygonal; they may fully or partially surround the barrel 402. A removable needle cover 408 protects the needle 400, shown in phantom.

The finger grips 406 are captured between the shelf 80 formed in the inner shell 50 and the terminus 210 of the cap 38. The conventional syringe 399 is therefore prevented from lateral movement inside the pig 121 during transit. The needle 400 and at least a portion of the barrel 402 are positioned in the hollow center section 94 of the base 46. At least a portion of the plunger 404 is positioned in the hollow center section 92 of the cap 38.

The closure structure is engaged and attaches the cap 38 to the base 46. The screw 82 threadably engages the hole 170 in the retaining ring 90. The head 81 of screw 82 is locked in the recess 160 of the small end 150 of the keyhole-shaped aperture 62. The screw 86 threadably engages the hole 174 in the retaining ring 90. The head 85 of screw 86 is locked in the recess 164 of the small end 154 of the keyhole-shaped aperture 66. The o-ring 72 is under compression and acts as a spring urging the cap 38 away from the base 46.

Method of Use Two-Part Pig

A prescription is called in, faxed in, or otherwise given to a radiopharmacy. The pharmacist enters the prescription in a computer and prints out the labels previously mentioned. A self-adhesive label can be attached to the pig in conventional fashion. In the alternative, a label can be attached to the pig with the flexible sleeve, without the need for adhesives. A separate label is affixed to a safety syringe or a conventional syringe. The syringe is filled with a radiopharmaceutical in accordance with the prescription. The filled syringe is assayed. In other words, the activity of the radiopharmaceutical in the syringe is measured in a dose calibrator to verify that it complies with the prescription. The filled syringe is put in the two-part pig and closed. The pig is wipe tested for contamination. If the pig passes the wipe test, it is placed in a delivery container.

The delivery containers used by some Mallinckrodt pharmacies have interior padding of rubber foam. Several pigs may be placed in a single delivery container. Before leaving the radiopharmacy, the delivery container and the pigs are wipe tested and surveyed. If the delivery container passes, a DOT label is affixed to the outside of the delivery container and it is delivered to a medical facility.

The pigs are then opened and the syringe is placed in an injection shield. The radiopharmaceutical is administered to the patient. Biodex produces a cylindrical injection shield that fits the B-D safety syringe and the Monoject® Safety Syringe. Cardinal Healthcare Ltd. recommends a clamp style safety syringe as disclosed in U.S. Pat. No. 6,162,198 for conventional syringes. After all the pigs have been opened and the radiopharmaceuticals have been administered, the DOT label is reversed. The reverse of the label clearly states BIOHAZARD and the following: “This package conforms to the conditions and limitations specified in 49 CFR 173.421 for radioactive material, excepted package—limited quantity of material, UN 2910.” The delivery case with the pigs and used syringes are then returned to the radiopharmacy. If a self-adhesive label has been applied to the base it is removed and discarded. If a non-adhesive label has been attached with the flexible sleeve, the sleeve is removed from the base and the label is discarded. The syringe is removed from the pig and placed in a disposal bin. The flexible sleeve is removed from the base and the label is thrown away. The pig is washed and dried. The pig is then ready to be reused.

Method of Use Three-Part Pig

The method of use for the three-part pig is the same as the two-part pig except an inner liner is inserted in the pig. A prescription is called in, faxed in, or otherwise given to a radiopharmacy. The pharmacist enters the prescription in a computer and prints out the labels previously mentioned. One label is attached to the pig with the flexible sleeve, and another is affixed to the safety syringe. The safety syringe or a conventional syringe is filled with a radiopharmaceutical in accordance with the prescription. The filled syringe is assayed. In other words, the activity of the radiopharmaceutical in the syringe is measured in a dose calibrator to verify that it complies with the prescription. The filled safety syringe is put in the three-part pig, which includes an inner liner in the base, never the cap. The pig is then closed. The pig is wipe tested for unwanted activity. If the pig passes the wipe test, it is placed in a delivery container.

The delivery containers used by some Mallinckrodt pharmacies have interior padding of rubber foam. A plurality of receptacles are formed in the foam and each is shaped to receive a pig. Several pigs may be placed in a single delivery container. Before leaving the radiopharmacy, the delivery container and the pigs are wipe tested and surveyed. If the delivery container passes, a DOT label is affixed to the outside of the delivery container. The DOT label contains the radioactivity symbol and the word Radioactive. The container is delivered to a medical facility.

The pigs are opened and typically, the syringe is placed in an injection shield. The radiopharmaceutical is then administered to the patient. The used syringe is then returned to the pig. After all the pigs have been opened and the radiopharmaceuticals have been administered, the DOT label is reversed. The reverse of the label clearly states BIOHAZARD and the following: “This package conforms to the conditions and limitations specified in 49 CFR 173.421 for radioactive material, excepted package—limited quantity of material, UN 2910.” The delivery case with the pigs and used syringes is then returned to the radiopharmacy. Each of the used syringes and the inner liners are removed from pigs and placed in a disposal bin. If a self-adhesive label has been applied to the base it is removed and discarded. If a non-adhesive label has been attached with the flexible sleeve, the sleeve is removed from the base and the label is discarded. The pig is washed and dried. The pig is then ready to be reused.

1. A pharmaceutical pig for transportation of a radiopharmaceutical in a syringe, the pig comprising:
an elongate base characterized by an inner and outer shell of the base completely enclosing a base shielding element;
an elongate cap removably attached to the base, the cap characterized by an inner and outer shell of the cap completely enclosing a cap shielding element, wherein the pig is characterized in that a portion of the cap shielding element overlaps a portion of the base shielding element.

2. The pig of claim 1, further including a flexible sleeve, at least a portion of which is transparent, removably disposed about the outer shell of the base.

3. The pig of claim 2, further including a label disposed between the flexible sleeve and the outer shell of the base.

4. The pig of claim 1, wherein the base shielding element and the cap shielding element are formed from lead.

5. The pig of claim 1, wherein the base shielding element and the cap shielding element are formed from a metallic-filled polymer composite material.

6. The pig of claim 1, wherein the inner and outer shell of the base are formed from stainless steel and are welded together to hermetically enclose the base shielding element to prevent contamination of the base shielding element, and the inner and outer shell of the cap are also formed from stainless steel and are welded together to hermetically enclose the cap shielding element to prevent contamination of the cap shielding element.

7. The pig of claim 6, further comprising a plurality of keyhole-shaped slots defined in the cap, wherein the slots are sized and arranged to receive a plurality of screws extending from the base to removably lock the cap to the base when at least one of the cap and base are rotated to engage the screws in the keyhole-shaped slots.

8. The pig of claim 1, wherein a hollow center section of the base is sized to accommodate a needle and at least a portion of a barrel of a syringe, and a hollow center section of the cap is sized to accommodate at least a portion of a plunger of the syringe.

9-10. (canceled)

11. The pig of claim 8, further including a flexible sleeve, at least a portion of which is transparent, to slip on and off at least a portion of the base to removably secure a label to the base.

12. The pig of claim 1, wherein the base shielding element is tapered near a syringe needle-accommodating portion of the pig, and the cap shielding element is of generally uniform thickness.

13. The pig of claim 12, further comprising means for enabling a bayonet-type interconnection of the cap and the base.

14. The pig of claim 1, further comprising means for enabling a bayonet-type interconnection of the cap and the base, wherein the inner and outer shell of the base are formed from stainless steel and are welded together to hermetically enclose the base shielding element to prevent contamination of the base shielding element and the inner and outer shell of the cap are also formed from stainless steel and are welded together to hermetically enclose the cap shielding element to prevent contamination of the cap shielding element.

15. The pig of claim 1, further comprising an elastomeric ring compressed between the cap and base, and a plurality of keyhole-shaped slots in the cap sized and aligned to receive a plurality of screws extending from the base to removably lock the cap to the base when the cap and base are rotated in opposite directions to engage the screws in the keyhole-shaped slots.

16. The pig of claim 1, wherein a portion of the base shielding element is disposed about a portion of the outer shell of the cap.

17. (canceled)

18. The pig of claim 1, wherein:
the inner and outer shell of the base are formed from stainless steel, and a hollow center section of the base is sized to accommodate a needle and at least a portion of a barrel of a syringe;
the inner and outer shell of the cap are formed from stainless steel, and a hollow center section of the cap is sized to accommodate at least a portion of a plunger of a syringe;
the cap shielding element has a generally uniform thickness;
the base shielding element is tapered in thickness near a portion of the hollow center section of the base that is sized to accommodate the needle; and
the base and cap include means for enabling a bayonet-type interconnection of the cap with the base.

19-30. (canceled)

29. An assembly for transporting a radiopharmaceutical, comprising:
a syringe having a needle, a barrel, and a plunger; and
a pharmaceutical pig comprising:
a base characterized by an inner and outer shell of the base completely enclosing a base shielding element;
an elongate cap removably attached to the base, the cap characterized by an inner and outer shell of the cap completely enclosing a cap shielding element; and
means for enabling a bayonet-type interconnection of the cap and the base,
wherein the syringe is disposed within an inner cavity of the pig, wherein a first portion of the cavity is defined by the base of the pig and is sized to accommodate the needle and at least a portion of the barrel of the syringe, and wherein a second portion of the cavity is defined by the cap of the pig and is sized to accommodate at least a portion of the plunger of the syringe.

30. The assembly of claim 29, wherein said means comprises:
a plurality of slots defined in the cap;
a plurality of screws extending from the base, wherein the screws are disposed in the slots defined in the cap; and
an elastomeric ring compressed between the cap and base, and disposed about at least a portion of the syringe.

31. The assembly of claim 29, further comprising:
a flexible sleeve, at least a portion of which is transparent, disposed about at least a portion of the outer shell of the base; and
a label disposed between said flexible sleeve and said outer shell of said base.

32. The assembly of claim 29, wherein the base shielding element is tapered near the needle of the syringe, the cap shielding element is of generally uniform thickness.

33. The assembly of claim 29, wherein a portion of the cap shielding element overlaps a portion of the base shielding element, and a portion of the inner shell of the base is disposed about a portion of the outer shell of the cap.
